(19) United States
(12) Patent Application Publication
(10) Pub. No.: US 2024/0238064 A1
(43) Pub. Date: Jul. 18, 2024

(54) PROTECTION OF CORONARY BLOOD VESSELS DURING CARDIAC PROCEDURES

(71) Applicant: Edwards Lifesciences Innovation (Israel) Ltd., Caesarea (IL)

(72) Inventors: Brian Patrick Murphy, Irvine, CA (US); Meena Francis, Trabuco Canyon, CA (US); Murrad Mirza Kazalbash, Tustin, CA (US); Lindsay Marie Hall, Murietta, GA (US); Mauricio Guerrero, Hawthorne, CA (US); Thomas V. Doherty, Irvine, CA (US)

(21) Appl. No.: 18/622,096

(22) Filed: Mar. 29, 2024

Related U.S. Application Data
(63) Continuation of application No. PCT/IB2022/058864, filed on Sep. 20, 2022.
(60) Provisional application No. 63/250,934, filed on Sep. 30, 2021.

(51) Int. Cl. A61B 90/00; A61B 34/00; A61F 2/24

(52) U.S. Cl. CPC A61B 90/08; A61B 34/73; A61F 2/2466; A61B 2090/08021; A61F 2220/0016

(57) ABSTRACT

A blood vessel is temporarily distanced from an anchor while the anchor is being driven into tissue of a subject. A delivery tool can be used to deliver the anchor to the tissue and to drive the anchor into the tissue. A vessel-protection device can be advanced transluminally into the blood vessel, and used to temporarily distance the blood vessel from the anchor while the anchor is driven into the tissue by the delivery tool. Other embodiments are also described.

### CROSS-REFERENCES TO RELATED APPLICATIONS

The present application is a Continuation of International Patent Application PCT/IB2022/058864 to Murphy et al., filed Sep. 20, 2022, and titled “Protection of coronary blood vessels during cardiac procedures,” which published as WO 2023/052901; which claims priority to and the benefit of Provisional U.S. Patent Application 63/250,934 to Murphy et al., filed Sep. 30, 2021, and titled “Protection of coronary blood vessels during cardiac procedures.”

Each of the above references is incorporated herein by reference in its entirety for all purposes.

### BACKGROUND

Some percutaneous (e.g., transluminal) cardiac interventions, such as annuloplasty techniques, involve anchoring one or more anchors to tissue of the heart. In some instances, the one or more anchors are driven into the tissue from within a chamber of the heart. In some such instances, the one or more anchors may be driven into the tissue in proximity to a blood vessel, such as a coronary blood vessel. For example, for some transluminal annuloplasty techniques, anchors are driven into the annulus of an atrioventricular valve being treated, in proximity to a blood vessel (e.g., a coronary artery) that extends alongside the annulus.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Systems, apparatuses, and techniques are disclosed for treating a subject, for example, a heart of a subject. For example, systems, apparatuses, and techniques are disclosed for protecting coronary blood vessels during performance of cardiac treatments, such as treatments that include driving a tissue anchor into tissue of the heart. The principles, systems, and devices can be adapted for other procedures as well and are not limited to cardiac procedures.

For some applications, a system is provided that includes (i) an anchor and a delivery tool therefor, and (ii) a vessel-protection device. The vessel-protection device is advanced into the blood vessel to be protected. While distancing the blood vessel from the anchor using the vessel-protection device disposed within the blood vessel, the delivery tool is used to drive the anchor into tissue of a subject. For some applications, the delivery tool is advanced into a chamber of the heart and is used to drive the anchor into the tissue from within the chamber. For example, the tissue may be tissue of an annulus of an atrioventricular valve, and the coronary blood vessel may extend alongside the annulus.

For some applications, the vessel-protection device distances the blood vessel from the anchor via magnetic repulsion (e.g., between the vessel-protection device and the anchor, or between the vessel-protection device and the delivery tool). This can distance the blood vessel and the anchor from each other by inhibiting the anchor/delivery tool from becoming too close to the blood vessel. In the example of an atrioventricular annuloplasty, this may translate into inhibiting the anchor from being anchored too close to the atrial wall within which a coronary artery is disposed.

For some applications, the vessel-protection device distances the blood vessel from the anchor mechanically, e.g., by applying mechanical force to part of the blood vessel. For example, the vessel-protection device may temporarily push part of the blood vessel (typically with tissue within which the vessel is disposed) away from the tissue to which the anchor is to be anchored, e.g., thereby providing more space for the anchor to be anchored. For example, the vessel-protection device can comprise a deflector that applies the mechanical force, e.g., by changing shape within the blood vessel. In the example of an atrioventricular annuloplasty, this may translate into pushing, away from the atrioventricular annulus, a part of the atrial wall within which a coronary artery is disposed.

There is provided, in accordance with some applications, a method for treating a subject having a blood vessel, the method comprising transluminally advancing a vessel-protection device into the blood vessel, and driving an anchor into tissue of the subject using a delivery tool while distancing the blood vessel from the anchor using the vessel-protection device disposed within the blood vessel.

In some applications, the method further comprises subsequently to driving the anchor into the tissue, removing the vessel-protection device from the blood vessel.

In some applications, the tissue is tissue of an atrium of a heart of the subject, and driving the anchor into the tissue comprises using a delivery tool disposed within the atrium to drive the anchor into the tissue. In some applications, the tissue is tissue of a ventricle of a heart of the subject, and driving the anchor into the tissue comprises using a delivery tool disposed within the ventricle to drive the anchor into the tissue.

In some applications, the anchor is a first anchor of an implant, and driving the anchor into the tissue comprises driving the first anchor of the implant into the tissue at a first site while distancing the blood vessel from the first anchor using the vessel-protection device disposed within the blood vessel in proximity to the first site. In some applications, the method further comprises subsequently to driving the first anchor into the tissue at the first site, moving the vessel-protection device within the blood vessel from being in proximity to the first site to being in proximity to a second site, and driving a second anchor of the implant into the tissue at the second site while distancing the blood vessel from the second anchor using the vessel-protection device disposed within the blood vessel in proximity to the second site.

In some applications, the implant is an adjustable implant including a tether coupled to the anchor, and wherein the method further comprises adjusting the implant by tensioning the tether subsequently to driving the anchor into the tissue.

In some applications, the tissue is tissue of an annulus of a heart valve, and the blood vessel is a coronary blood vessel in proximity to the annulus, and wherein driving the anchor into the tissue comprises driving the anchor into the annulus.

In some applications, the anchor is an anchor of an annuloplasty implant, and wherein driving the anchor into the annulus comprises driving the anchor of the annuloplasty implant into the annulus.

In some applications, driving the anchor into the tissue comprises driving the anchor into the tissue using a delivery tool while distancing the blood vessel from the anchor by applying a mechanical force using the vessel-protection device disposed within the blood vessel.

In some applications, the vessel-protection device includes a deflector, and wherein applying the mechanical force using the vessel-protection device comprises temporarily pushing a part of the blood vessel away from the tissue using the deflector.

In some applications, pushing the part of the blood vessel away from the tissue using the deflector comprises pushing the part of the blood vessel away from the tissue by temporarily transitioning the deflector into a deflecting state in which the deflector bulges away from the tissue.

In some applications, the deflector is biased toward assuming the deflecting state, and transluminally advancing the vessel-protection device into the blood vessel comprises transluminally advancing the deflector into the blood vessel while the deflector is constrained in a non-deflecting state, and transitioning the deflector into the deflecting state comprises eliminating the constraint such that the deflector automatically responsively transitions toward the deflecting state.

In some applications, the vessel-protection device includes a tether that extends proximally from the deflector, and transitioning the deflector into the deflecting state comprises transitioning the deflector into the deflecting state by tensioning the tether.

In some applications, transitioning the deflector into the deflecting state comprises transitioning the deflector into the deflecting state by tensioning the tether such that the deflector curves.

In some applications, transitioning the deflector into the deflecting state comprises transitioning the deflector into the deflecting state by tensioning the tether such that the deflector articulates.

In some applications, driving the anchor into the tissue comprises driving the anchor into the tissue using a delivery tool while distancing the blood vessel via magnetic repulsion using the vessel-protection device disposed within the blood vessel.

In some applications, distancing the blood vessel via magnetic repulsion comprises distancing the blood vessel via magnetic repulsion between the vessel-protection device disposed within the blood vessel and the anchor disposed within the chamber.

In some applications, distancing the blood vessel via magnetic repulsion comprises distancing the blood vessel via magnetic repulsion between the vessel-protection device disposed within the blood vessel and the delivery tool disposed within the chamber.

The foregoing method(s) can be performed on a living animal or on a simulation, such as on a cadaver, cadaver heart, simulator (e.g., with the body parts, heart, tissue, etc. being simulated), etc.

There is further provided, in accordance with some applications, a system for treating a subject, the system comprising an implant, comprising a tissue anchor, a delivery tool configured to deliver the tissue anchor to tissue of the subject and drive the tissue anchor into the tissue, and a vessel-protection device configured to be advanced transluminally into a blood vessel and to temporarily distance the blood vessel from when the anchor driven into the tissue by the delivery tool.

In some applications, the implant comprises multiple tissue anchors, the tissue anchor being a first tissue anchor of the multiple tissue anchors, the delivery tool is configured to sequentially drive each of the multiple anchors into respective multiple sites of the tissue, and the vessel-protection device is configured to be movable within the blood vessel such that, for each of the multiple anchors sequentially, the vessel-protection device is configured to temporarily distance the blood vessel from the anchors during the driving, by the delivery tool, of the anchor into the respective site of the tissue.

In some applications, the implant is an adjustable implant comprising a tether coupled to the anchor.

In some applications, the tissue is tissue of an atrium of the heart, and wherein the delivery tool is configured to transluminally advance the anchor to the atrium and, from within the atrium, to drive the anchor into the tissue.

In some applications, the tissue is tissue of a ventricle of the heart, and wherein the delivery tool is configured to transluminally advance the anchor to the ventricle and, from within the ventricle, to drive the anchor into the annulus.

In some applications, the heart has a valve adjacent the chamber, the valve having an annulus, and the blood vessel being in proximity to the annulus, the delivery tool is configured to, from within the chamber, drive the anchor into the annulus, and the vessel-protection device is configured to temporarily distance the blood vessel from the anchor during the driving, by the delivery tool, of the anchor into the annulus.

In some applications, the implant is an annuloplasty implant.

In some applications, the vessel-protection device is configured to temporarily distance the blood vessel from the anchor via a mechanical force.

In some applications, the vessel-protection device comprises a deflector that is configured to temporarily push a part of the blood vessel away from the tissue.

In some applications, the deflector is configured to temporarily push the part of the blood vessel away from the tissue by, while disposed within the part of the blood vessel, temporarily transitioning toward a deflecting state in which the deflector bulges away from the tissue.

In some applications, the deflector is configured to be advanced while constrained in a non-deflecting state and is biased to transition toward the deflecting state automatically in response to elimination of the constraint.

In some applications, the deflector comprises a ribbon that is configured to be advanced while constrained in the non-deflecting state and is biased to transition toward the deflecting state automatically in response to elimination of the constraint.

In some applications, the deflector comprises a wire that is configured to be advanced while constrained in the non-deflecting state and is biased to transition toward the deflecting state automatically in response to elimination of the constraint.

In some applications, the vessel-protection device comprises a tether that extends proximally from the deflector, and wherein the deflector is configured to transition toward the deflecting state in response to tensioning of the tether.

In some applications, the deflector comprises a tube, the tether is fixed to a distal part of the deflector, and extends proximally through the tube, and the deflector is configured to transition toward the deflecting state by the tube curving in response to tensioning of the tether.

In some applications, the deflector comprises multiple articulated sections in series, the tether is fixed to a distal part of the deflector, and extends proximally along the deflector, and the deflector is configured to transition toward the deflecting state by the articulated sections articulating in response to tensioning of the tether.

In some applications, the vessel-protection device is configured to temporarily distance the blood vessel from the anchor via magnetic repulsion.

In some applications, the vessel-protection device is configured to temporarily distance the blood vessel from the anchor via magnetic repulsion between the vessel-protection device and the implant.

In some applications, the vessel-protection device is configured to temporarily distance the blood vessel from the anchor via magnetic repulsion between the vessel-protection device and the delivery tool.

In some applications, at least one component selected from the group consisting of the implant, the delivery tool, and the vessel-protection device comprises an electromagnet, and at least another component selected from the group comprises a ferrous element (e.g., a ferrous wire, a ferrous ring, a ferrous coating, a ferrous strip, a ferrous patch, a ferrous tip, a ferrous, coil, a ferrous shape, a ferrous rod, etc.)

In some applications, the one component is the vessel-protection device. In some applications, the other component is the implant. In some applications, the other component is the anchor of the implant. In some applications, the other component is the delivery tool.

In some applications, the delivery tool comprises a flexible tube and an anchor driver disposed within the flexible tube, and the other component is the flexible tube of the delivery tool, the ferrous element being disposed at a distal part of the flexible tube.

In some applications, the delivery tool comprises a flexible tube and an anchor driver disposed within the flexible tube, and the other component is the anchor driver, the ferrous element being disposed at a distal part of the anchor driver.

There is provided, in accordance with some applications, a method, for treating a heart of a subject, the heart having a chamber and a coronary blood vessel in adjacent the chamber, the method including transluminally advancing a vessel-protection device into the coronary blood vessel; and transluminally advancing a delivery tool into the chamber. The method can further include driving an anchor into tissue of the heart using the delivery tool disposed within the chamber while distancing the coronary blood vessel from the anchor using the vessel-protection device disposed within the coronary blood vessel.

In some applications, the method further includes, subsequently to driving the anchor into the tissue, removing the vessel-protection device from the coronary blood vessel.

In some applications, the chamber is an atrium of the heart, and driving the anchor into the tissue using the delivery tool disposed within the chamber includes driving the anchor into the tissue using the delivery tool disposed within the atrium.

In some applications, the chamber is a ventricle of the heart, and driving the anchor into the tissue using the delivery tool disposed within the chamber includes driving the anchor into the tissue using the delivery tool disposed within the ventricle.

In some applications, the anchor is a first anchor of an implant, and driving the anchor into the tissue includes driving the first anchor of the implant into the tissue at a first site while distancing the coronary blood vessel from the first anchor using the vessel-protection device disposed within the coronary blood vessel in proximity to the first site.

In some applications, the method further includes subsequently to driving the first anchor into the tissue at the first site, moving the vessel-protection device within the coronary blood vessel from being in proximity to the first site to being in proximity to a second site, and driving a second anchor of the implant into the tissue at the second site while distancing the coronary blood vessel from the second anchor using the vessel-protection device disposed within the coronary blood vessel in proximity to the second site.

In some applications, the implant is an adjustable implant including a tether coupled to the anchor, and the method further includes adjusting the implant by tensioning the tether subsequently to driving the anchor into the tissue.

In some applications, the heart has a valve adjacent the chamber, the valve having an annulus, and the coronary blood vessel being in proximity to the annulus, the tissue is tissue of the annulus, and driving the anchor into the tissue includes driving the anchor into the annulus.

In some applications, the anchor is an anchor of an annuloplasty implant, and driving the anchor into the annulus includes driving the anchor of the annuloplasty implant into the annulus.

In some applications, driving the anchor into the tissue includes driving the anchor into the tissue using the delivery tool disposed within the chamber while distancing the coronary blood vessel from the anchor by applying a mechanical force using the vessel-protection device disposed within the coronary blood vessel.

In some applications, the vessel-protection device includes a deflector, and applying the mechanical force using the vessel-protection device includes temporarily pushing a part of the coronary blood vessel away from the tissue using the deflector.

In some applications, pushing the part of the coronary blood vessel away from the tissue using the deflector includes pushing the part of the coronary blood vessel away from the tissue by temporarily transitioning the deflector into a deflecting state in which the deflector bulges away from the tissue.

In some applications, the deflector is biased toward assuming the deflecting state, transluminally advancing the vessel-protection device into the coronary blood vessel includes transluminally advancing the deflector into the coronary blood vessel while the deflector is constrained in a non-deflecting state, and transitioning the deflector into the deflecting state includes eliminating the constraint such that the deflector automatically responsively transitions toward the deflecting state.

In some applications, the vessel-protection device includes a tether that extends proximally from the deflector, and transitioning the deflector into the deflecting state includes transitioning the deflector into the deflecting state by tensioning the tether.

In some applications, transitioning the deflector into the deflecting state includes transitioning the deflector into the deflecting state by tensioning the tether such that the deflector curves.

In some applications, transitioning the deflector into the deflecting state includes transitioning the deflector into the deflecting state by tensioning the tether such that the deflector articulates.

In some applications, driving the anchor into the tissue includes driving the anchor into the tissue using the delivery tool disposed within the chamber while distancing the coronary blood vessel via magnetic repulsion using the vessel-protection device disposed within the coronary blood vessel.

In some applications, distancing the coronary blood vessel via magnetic repulsion includes distancing the coronary blood vessel via magnetic repulsion between the vessel-protection device disposed within the coronary blood vessel and the anchor disposed within the chamber.

In some applications, distancing the coronary blood vessel via magnetic repulsion includes distancing the coronary blood vessel via magnetic repulsion between the vessel-protection device disposed within the coronary blood vessel and the delivery tool disposed within the chamber.

The foregoing method(s) can be performed on a living animal or on a simulation, such as on a cadaver, cadaver heart, simulator (e.g., with the body parts, heart, tissue, etc. being simulated), etc.

There is further provided, in accordance with some applications, a system for treating a heart of a subject, the heart having a chamber and a coronary blood vessel adjacent the chamber, the system including and implant, a delivery tool, and a vessel-protection device. The implant can include a tissue anchor. The delivery tool can be configured to transluminally advance the anchor to the chamber and to, from within the chamber, drive the anchor into tissue of the heart. The vessel-protection device can be configured to be advanced transluminally into the coronary blood vessel and to temporarily distance the coronary blood vessel from the anchor during the driving, by the delivery tool, of the anchor into the tissue.

In some applications, the implant includes multiple tissue anchors, the tissue anchor being a first tissue anchor of the multiple tissue anchors, the delivery tool is configured to sequentially drive each of the multiple anchors into respective multiple sites of the tissue, and the vessel-protection device is configured to be movable within the coronary blood vessel such that, for each of the multiple anchors sequentially, the vessel-protection device is configured to temporarily distance the coronary blood vessel from the anchors during the driving, by the delivery tool, of the anchor into the respective site of the tissue.

In some applications, the implant is an adjustable implant including a tether coupled to the anchor.

In some applications, the chamber is an atrium of the heart, and the delivery tool is configured to transluminally advance the anchor to the atrium and, from within the atrium, to drive the anchor into the tissue.

In some applications, the chamber is a ventricle of the heart, and the delivery tool is configured to transluminally advance the anchor to the ventricle and, from within the ventricle, to drive the anchor into the annulus.

In some applications, the heart has a valve adjacent the chamber, the valve having an annulus, and the coronary blood vessel being in proximity to the annulus, and the delivery tool is configured to, from within the chamber, drive the anchor into the annulus, and the vessel-protection device is configured to temporarily distance the coronary blood vessel from the anchor during the driving, by the delivery tool, of the anchor into the annulus.

In some applications, the implant is an annuloplasty implant.

In some applications, the vessel-protection device is configured to temporarily distance the coronary blood vessel from the anchor via a mechanical force.

In some applications, the vessel-protection device includes a deflector that is configured to temporarily push a part of the coronary blood vessel away from the tissue.

In some applications, the deflector is configured to temporarily push the part of the coronary blood vessel away from the tissue by, while disposed within the part of the coronary blood vessel, temporarily transitioning toward a deflecting state in which the deflector bulges away from the tissue.

In some applications, the deflector is configured to be advanced while constrained in a non-deflecting state and is biased to transition toward the deflecting state automatically in response to elimination of the constraint.

In some applications, the deflector includes a ribbon that is configured to be advanced while constrained in the non-deflecting state and is biased to transition toward the deflecting state automatically in response to elimination of the constraint.

In some applications, the deflector includes a wire that is configured to be advanced while constrained in the non-deflecting state and is biased to transition toward the deflecting state automatically in response to elimination of the constraint.

In some applications, the vessel-protection device includes a tether that extends proximally from the deflector, and the deflector is configured to transition toward the deflecting state in response to tensioning of the tether.

In some applications, the deflector includes a tube. In some applications, the tether is fixed to a distal part of the deflector, and extends proximally through the tube. In some applications, the deflector is configured to transition toward the deflecting state by the tube curving in response to tensioning of the tether.

In some applications, the deflector includes multiple articulated sections in series. In some applications, the tether is fixed to a distal part of the deflector, and extends proximally along the deflector. In some applications, the deflector is configured to transition toward the deflecting state by the articulated sections articulating in response to tensioning of the tether.

In some applications, the vessel-protection device is configured to temporarily distance the coronary blood vessel from the anchor via magnetic repulsion.

In some applications, the vessel-protection device is configured to temporarily distance the coronary blood vessel from the anchor via magnetic repulsion between the vessel-protection device and the implant.

In some applications, the vessel-protection device is configured to temporarily distance the coronary blood vessel from the anchor via magnetic repulsion between the vessel-protection device and the delivery tool.

In some applications, at least one component (e.g., a first component) selected from the group consisting of the implant, the delivery tool, and the vessel-protection device includes an electromagnet. In some applications, at least another component (e.g., a second component) selected from the group includes a ferrous element.

In some applications, the one component is the vessel-protection device.

In some applications, the other component is the implant.

In some applications, the other component is the anchor of the implant.

In some applications, the other component is the delivery tool.

In some applications, the delivery tool includes a flexible tube and an anchor driver disposed within the flexible tube, and the other component is the flexible tube of the delivery tool, the ferrous element being disposed at a distal part of the flexible tube.

In some applications, the delivery tool includes a flexible tube and an anchor driver disposed within the flexible tube, and the other component is the anchor driver, the ferrous element being disposed at a distal part of the anchor driver.

There is further provided, in accordance with some applications, a system for treating a heart of a subject, the heart having a chamber and a coronary blood vessel adjacent the chamber, the system including a tissue anchor; and anchor driver, and a position-detecting assembly. The anchor driver can further include a flexible tube that is transluminally advanceable into the chamber.

The anchor driver can include a magnet. The magnet can be disposed at a distal portion of the tube.

In some applications, the anchor driver can further include a rod that is configured to drive the anchor out of the distal portion of the tube and into tissue of the heart.

The position-detecting assembly can include a microelectromechanical system magnetic field sensor (MEMS-MFS), transluminally advanceable into the heart, and configured to sense a magnetic field of the magnet. The position-detecting assembly can further include circuitry that is configured to derive, from the sensed magnetic field, information regarding a position of the distal portion of the tube within the heart.

In some applications, the position-detecting assembly includes an interface, configured to provide an indication of the derived information.

In some applications, the information is information regarding a proximity of the distal portion of the tube to the coronary blood vessel, and the position-detecting assembly is configured to derive, from the sensed magnetic field, the information regarding the proximity of the distal portion of the tube to the coronary blood vessel.

In some applications, the MEMS-MFS is coupled to a distal portion of the rod.

In some applications, the system further includes a longitudinal member that is transluminally advanceable into the coronary blood vessel, and the MEMS-MFS is configured to sense, from within the chamber, distortion of the magnetic field by the longitudinal member within the coronary blood vessel. In some applications, the position-detecting assembly is configured to derive the information from the sensed distortion of the magnetic field.

In some applications, the longitudinal member is a wire. In some applications, the longitudinal member is one or more of a rod, wire, extension, tube, hypotube, strip, ribbon, shaft, etc.

In some applications, the MEMS-MFS is in electronic communication with the circuitry via the rod.

In some applications, the system further includes a longitudinal member that is transluminally advanceable into the coronary blood vessel, and the longitudinal member is configured to, from within the coronary blood vessel, facilitate the position-detecting assembly to derive the information.

In some applications, the information is information regarding a proximity between (i) the distal portion of the tube within the chamber and (ii) the longitudinal member within the coronary blood vessel, and the longitudinal member is configured to, from within the coronary blood vessel, facilitate the position-detecting assembly to derive the information regarding the proximity between (i) the distal portion of the tube within the chamber and (ii) the longitudinal member within the coronary blood vessel.

In some applications, the MEMS-MFS is coupled to the longitudinal member and is configured to sense the magnetic field from within the coronary blood vessel.

In some applications, the MEMS-MFS is in electronic communication with the position-detecting assembly via the longitudinal member.

In some applications, the MEMS-MFS is coupled to the anchor driver, and is configured to sense, from within the chamber, distortion of the magnetic field by the longitudinal member within the coronary blood vessel, and the position-detecting assembly is configured to derive the information from the sensed distortion of the magnetic field.

In some applications, the longitudinal member is a wire. In some applications, the longitudinal member is one or more of a rod, wire, extension, tube, hypotube, strip, ribbon, shaft, etc.

In some applications, the MEMS-MFS is coupled to a distal portion of the rod.

In some applications, the MEMS-MFS is in electronic communication with the position-detecting assembly via the rod.

There is further provided, in accordance with some applications, a system for treating a heart of a subject, the heart having a chamber and a coronary blood vessel adjacent the chamber, the system including a tissue anchor; an anchor driver; and a vessel-protection device.

The anchor driver can include a flexible tube, having a distal portion that has a tube axis and that is transluminally advanceable into the chamber.

The anchor driver can also include a first magnet, disposed at the distal portion of the tube, and having a first magnetic axis that is substantially parallel with the tube axis.

The system can also include a rod, configured to drive the anchor out of the distal portion of the tube within the chamber and into tissue of the heart.

In some applications, the vessel-protection device can include a longitudinal member, having a distal portion that has a longitudinal axis and that is transluminally advanceable into the coronary blood vessel.

The vessel-protection device can further include a second magnet, disposed at the distal portion of the longitudinal member, and having a second magnetic axis that is substantially orthogonal to the longitudinal axis.

The system can be configured such that upon approximation of the distal portion of the tube within the chamber with the distal portion of the longitudinal member within the coronary blood vessel, a magnetic force between the first magnet and the second magnet biases the tube axis toward being parallel with the second magnetic axis.

In some applications, the tube is sufficiently flexible to bend in response to the magnetic force.

In some applications, at least one magnet selected from the group consisting of the first magnet and the second magnet is an electromagnet.

In some applications, at least another magnet selected from the group consisting of the first magnet and the second magnet is a permanent magnet.

In some applications, both the first magnet and the second magnet are electromagnets.

In some applications, the vessel-protection device includes multiple second magnets, the second magnet being one of the multiple second magnets, and each of the multiple second magnets is disposed at the distal portion of the longitudinal member, and each has a respective second magnetic axis that is substantially orthogonal to the longitudinal axis.

There is further provided, in accordance with some applications, a method, for treating a heart of a subject, the heart having a chamber and a coronary blood vessel in adjacent the chamber, the method including transluminally advancing an anchor driver into the chamber. The anchor driver can include a flexible tube; a first magnet, disposed at the distal portion of the tube, and a rod. The flexible tube can have a distal portion that has a tube axis, is transluminally advanceable into the chamber, and has a distal opening. The rod can be disposed or disposable within the tube. The first magnet can have a first magnetic axis that is substantially parallel with the tube axis.

In some applications, the method further includes transluminally advancing a vessel-protection device into the coronary blood vessel. The vessel-protection device can include a longitudinal member, and a second magnet. The longitudinal member can have a distal portion that has a longitudinal axis and that is transluminally advanceable into the coronary blood vessel. The second magnet can be disposed at the distal portion of the longitudinal member and can have a second magnetic axis that is substantially orthogonal to the longitudinal axis.

In some applications, the method can further include bringing the distal portion of the tube, within the chamber, into proximity with the vessel-protection device within the coronary blood vessel, such that a magnetic force between the first magnet and the second magnet biases the tube axis toward being parallel with the second magnetic axis. The method can further include, while the magnetic force continues to bias the tube axis toward being parallel with the second magnetic axis, driving an anchor out of the distal opening and into tissue of the heart.

The foregoing methods can be performed on a living animal or on a simulation, such as on a cadaver, cadaver heart, simulator (e.g., with the body parts, heart, tissue, etc. being simulated), etc.

The present invention will be more fully understood from the following detailed description of applications thereof, taken together with the drawings, in which:

### BRIEF DESCRIPTION OF THE DRAWINGS

FIGS. 1A-E, 2A-C, 3A-E, and 4A-C are schematic illustrations of example systems for protection of coronary blood vessels, in accordance with some applications;

FIGS. 5A-E and 6A-C are schematic illustrations of example heart valve repair systems, in accordance with some applications;

FIGS. 7A-D are schematic illustrations of an example heart valve repair system, in accordance with some applications;

FIGS. 8A-D and 9A-D are schematic illustrations of example heart valve repair systems, in accordance with some applications; and

FIGS. 10-11 are schematic illustrations of example heart valve repair systems, in accordance with some applications.

### DETAILED DESCRIPTION

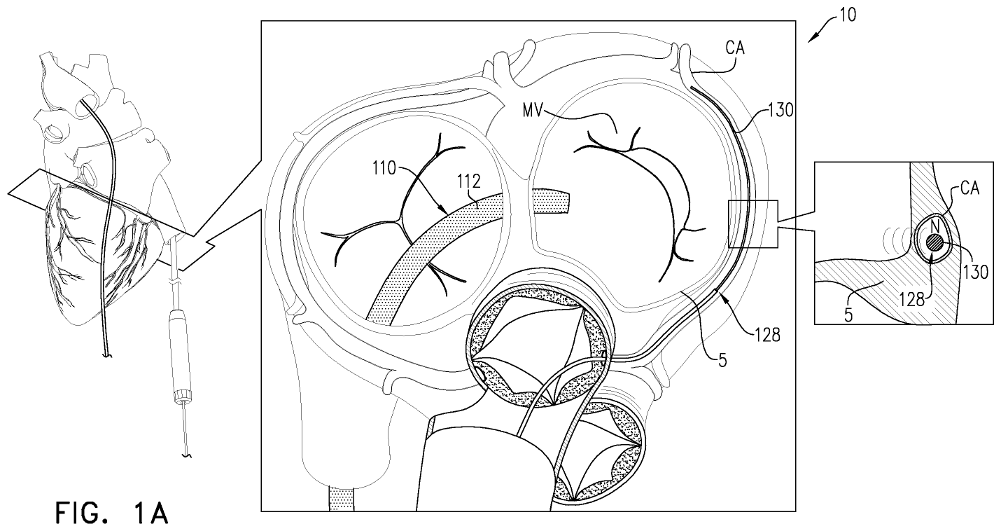

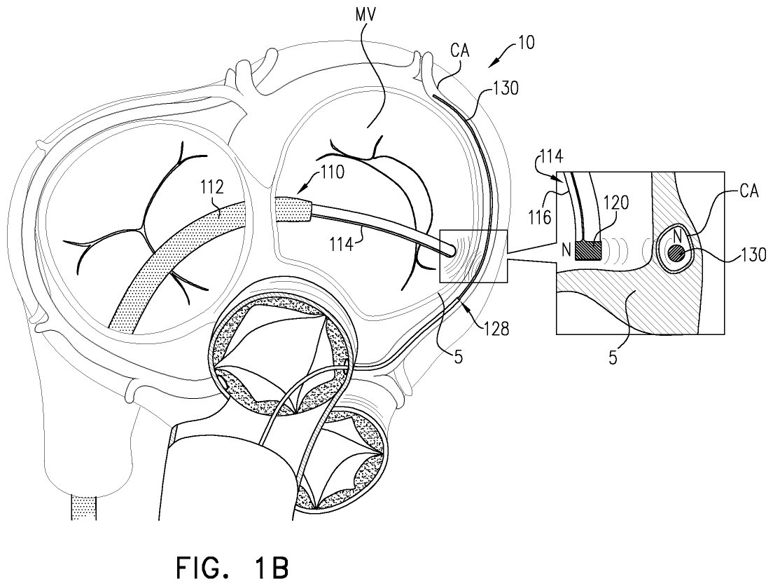

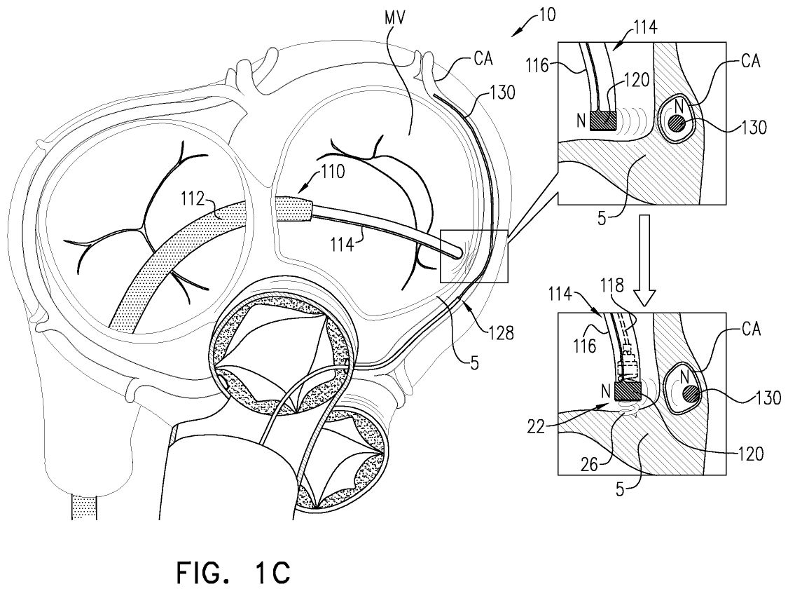

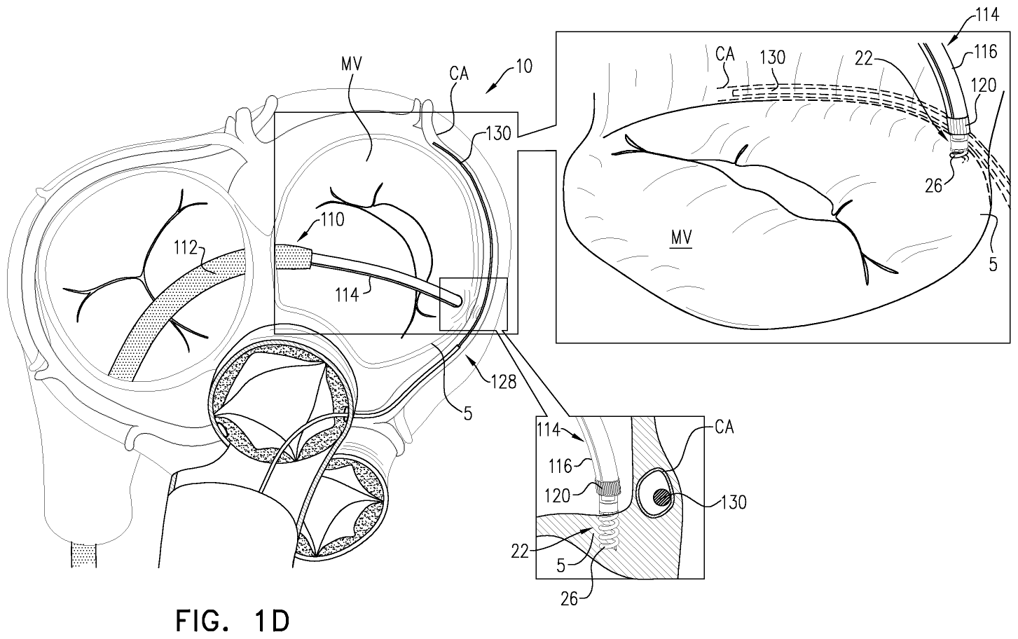

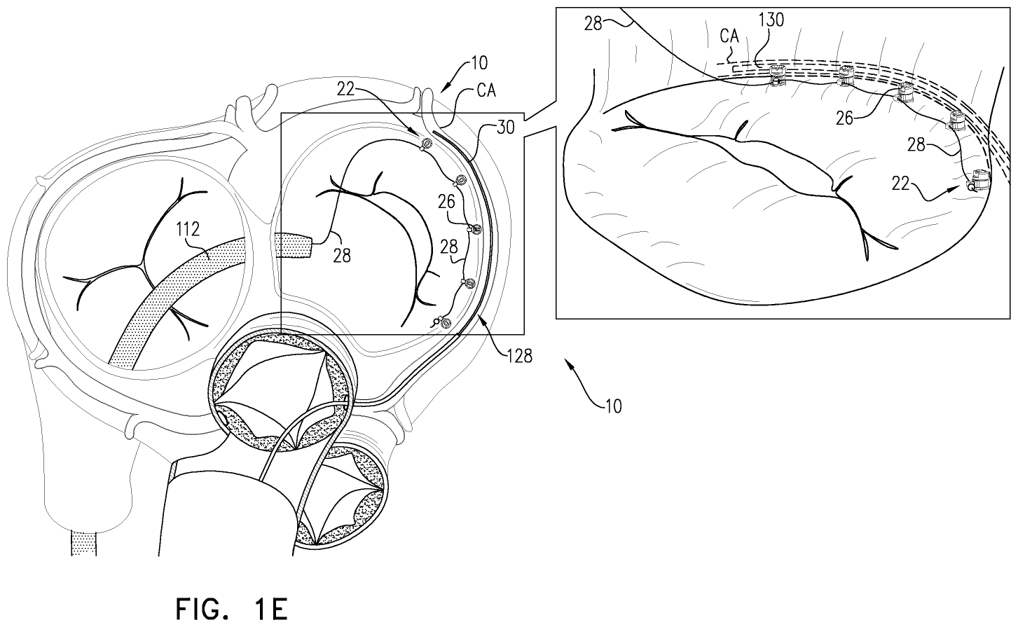

Reference is made to FIGS. 1A-E, 2A-C, 3A-E, and 4A-C, which are schematic illustrations of example systems for protection of coronary blood vessels, in accordance with some applications. These systems can be used for protection of coronary blood vessels during anchoring of anchors to tissue of the heart. These systems operate primarily via magnetic means.

FIGS. 1A-E show a heart valve repair system 10, in accordance with some applications, for repairing a heart valve. System 10 comprises a delivery tool 110 for navigating an implant 22 toward a valve of a heart to be treated. Delivery tool 110 can comprise a catheter 112, and/or an anchor driver 114 that can be advanced through the catheter. Example implants are shown and described herein, but the implant could be configured in a variety of different ways for a variety of different purposes. In other words, the systems, methods, components, devices, etc. herein can be used to help anchor a variety of different implants regardless of whether those implants are expressly shown herein.

Implant 22 comprises one or more tissue anchors 26. In the example shown, implant 22 comprises multiple tissue anchors that are slidably coupled to (e.g., threaded onto) a tether 28. However, implant 22 may comprise only one tissue anchor, and/or may not be coupled to a tether. Anchor driver 114 is configured to reversibly engage tissue anchors 26, and to drive them into tissue of, or adjacent to, the mitral valve. Driver 114 can comprise a flexible tube 116 through which implant 22 (e.g., anchors 26 thereof) can be advanced, (e.g., as shown), and can also comprise a rod 118 that, for each of the anchors, is reversibly couplable to the anchor, and is configured to advance the anchor through the tube and drive the anchor into the tissue. For simplicity, rod 118 is shown only in FIG. 1C, where it is shown in phantom, representing its presence inside tube 116. Catheter 112 can be transluminally (e.g., transfemorally, transeptally, transvascularly, etc.) advanceable to the heart.

System 10 further comprises a vessel-protection device 128 configured to protect a coronary artery (CA) from damage during anchoring of tissue anchor 26, e.g., by reducing a likelihood that anchor 26 will contact and/or penetrate the coronary artery.

Tissue anchors 26 are configured to be anchorable in tissue, such as the annulus of the valve, to secure implant 22 to the tissue. For example, implant 22 can be an annuloplasty implant comprising multiple anchors 26 slidably coupled to a tether 28, tensioning of the tether contracting the tissue in order to repair a leaky mitral valve, e.g., as described in one or more of the examples disclosed in U.S. Pat. No. 9,949,828 to Sheps et al., US Patent Application Publication 2020/0015971 to Brauon et al., US Patent Application Publication 2021/0145584 to Kasher et al., and/or PCT Application Publication WO 2022/172149 to Shafigh et al., each of which is incorporated herein by reference for all purposes. The method(s) described in these references and herein can be performed on a living animal or on a simulation, such as on a cadaver, cadaver heart, simulator (e.g., with the body parts, heart, tissue, etc. being simulated), etc. mutatis mutandis.

Vessel-protection device 128 is designed to distance the coronary artery (CA) from implant 22, in particular anchors 26, so as to mitigate and preferably prevent any possible damage to the coronary artery. Vessel-protection device 128 utilizes a magnetic feature to provide a repelling force to provide such distancing. In some implementations, the magnetic feature can be used to guide and/or bias implant 22 to a desired location in the mitral valve (MV). System 10 thus utilizes a technique wherein each of anchors 26 is distanced from the coronary artery (CA) by way of magnetically repelling the anchor from vessel-protection device 128.

Device 128 comprises a magnetic element 130 such as an electromagnet or a permanent magnet, and can be in the form of a wire, e.g., as shown, or in any other form. Magnetic element 130 is configured, in combination with a repellable part of delivery tool 110 and/or implant 22, to cause magnetic repulsion between the magnetic element and the repellable part. For example, the repellable part can comprise a magnet or a ferrous metal. For applications in which the repellable part is part of implant 22, the repellable part can be part of anchor 26 (e.g., a head thereof), and/or part of a spacer disposed on tether 28 between anchors. Although not shown, such a spacer can be threaded onto tether 28, can be tubular, and/or can be as described in US patent Application Publication 2021/0145584 to Kasher et al., and/or PCT Application Publication WO 2022/172149 to Shafigh et al., each of which is incorporated herein by reference in its entirety.

For applications in which the repellable part is part of tool 110, the repellable part can be part of driver 114 (e.g., a distal part of tube 116 or rod 118). In the example shown, the repellable part is embodied as a repellable part 120 at a distal part of tube 116.

Tissue anchors 26 are shown as screw-in tissue anchors, but can alternatively be any of a variety of types including darts or staples, and are not described in detail.

The respective polarities of part 120 and magnetic element 130 can be configured so as to repel each other. For example, part 120 and magnetic element 130 may have the same polarity as each other. As such, when an anchor 26 is navigated to an intended anchoring position by delivery tool 110, the magnetic repulsion inhibits part 120—and thereby the distal part of tube 116—from approaching (e.g., becoming too close to) magnetic element 130, and thereby coronary artery (CA). This may advantageously protect the coronary artery from damage (e.g., perforation) by anchor 26. The device 128 may control, choose, select or otherwise adjust or set the strength of the magnetization so as to set the distance between anchor 26 and magnetic element 130.

FIG. 1B shows driver 114 (e.g., tube 116 thereof) disposed within the left atrium, in the vicinity of the mitral valve (MV). Arcs are used to schematically illustrate the respective magnetic fields of part 120 and element 130. As driver 114 approaches an annulus 5 of the mitral valve, magnetic repulsion between magnetic element 130 and part 120 begins to strengthen (FIG. 1C). The lower enlargement of FIG. 1C shows an example of anchor 26 being driven into the annulus spaced apart from the coronary artery—which may be the closest distance to the coronary artery allowed by this magnetic repulsion.

For some applications, and as shown, as implant 22 and/or driver 24 approaches coronary artery CA, the magnetic repulsion repels a portion of magnetic element 130 against the far wall of the coronary artery (rightward in the lower enlargement of FIG. 3). For some such applications, this movement of magnetic element 130 may also push a portion of the coronary artery (e.g., with the portion of the atrial wall in which the coronary artery is disposed) away from the anchoring site, thus protectively moving the coronary artery away from the site at which anchor 26 will be anchored. FIG. 1D shows anchor 26 having been driven completely into tissue of annulus 5.

For some applications, and as shown, additional anchors 26 of implant 22 are subsequently driven into annulus 5. FIG. 1E shows implant 22 having been anchored by five anchors 26. In the example shown, implant 22 comprises multiple anchors 26 and a tether 28, to which the anchors are slidably coupled (e.g., threaded onto). Once implant 22 has been implanted, device 128 can be withdrawn. For some applications, either before or after withdrawal of device 128, implant 22 can be adjusted to reduce regurgitation of the mitral valve (MV), for example by tensioning tether 28 in order to draw anchors 26 closer together.

FIGS. 2A-C are schematic illustrations of a heart valve repair system 11 comprising a vessel-protection device 178, in accordance with some applications. System 11, device 178, and/or techniques for use therewith can be as described hereinabove for system 10 and/or device 128, except that device 178 comprises a plurality of discrete magnets 32, rather than a continuous wire that extends along the coronary artery. For example, device 178 can comprise a magnet 32 for each anchor 26 that is to be implanted. Magnets 32 are illustrated as or sleeve-like sections or cylindrical attachments to a wire 180 of device 178, but it is to be understood that the scope of the disclosure includes other configurations. Device 178 is threaded into the coronary artery (CA) in order to position magnets 32 prior to anchoring of implants 22.

In FIG. 2A, driver 114 has been directed into the left atrium, but is still well distanced from vessel-protection device 178 and its magnets 32. As driver 114 approaches the coronary artery (FIG. 2B), magnetic repulsion between a given magnet 32 and part 120 increases. As such, anchor 26 is inhibited from coming into potentially detrimental proximity of the coronary artery. In FIG. 6C, all of anchors 26 have been driven into annulus 5 of mitral valve (MV), each opposite a corresponding magnet 32.

For some applications, magnets 32 are electromagnets, which are temporarily magnetized. For some such applications, each of magnets 32 is independently addressable. Although this is represented in FIG. 2B by only the proximal-most of magnets 32 being illustrated with a magnetic field, it is to be noted that the scope of the present disclosure includes using this independent addressability to activate more than one of magnets 32 at a time. The device 178 and/or system 11 may independently control, activate or adjust the strength of magnetization of each of the magnets 32 to precisely control the direction, redirection, repulsion or movement of a corresponding anchor.

For some applications, such independent addressing is used solely for magnetic repulsion. However, for some applications, for the anchoring of a given anchor, a first subset of magnets 32 can be activated to induce magnetic repulsion of part 120 while a second subset of the magnets can be activated to induce magnetic attraction of part 120, e.g., by the first and second subsets having different polarities. This can advantageously further facilitate placement of anchors 26, e.g., by biasing part 120 toward a specific location rather than by merely repelling it.

FIGS. 3A-E are schematic illustrations of a heart valve repair system 12 comprising a vessel-protection device 228, in accordance with some applications. System 12, device 228, and/or techniques for use therewith can be as described hereinabove for system 10, system 11, device 128, and/or device 178, except that whereas devices 128 and 178 can be left stationary during implantation of implant 22, device 228 is configured to be moved throughout implantation, e.g., for the anchoring of each anchor 26. For some applications, device 228 comprises a single discrete magnet. For some applications, device 228 comprises several magnets. Device 228 can be advanced into the coronary artery (CA) such that magnet 32 is firstly located in a position opposite/corresponding to where the first anchor 26 is to be driven into the annulus of mitral valve (MV). Magnet 32 can be disposed at the distal end of device 228, as illustrated. In some applications, magnet 32 can be disposed at any suitable location along device 228.

In FIG. 3A, the distal end of tube 116 has been directed into the area of mitral valve (MV) and is beginning to approach magnet 32 of vessel-protection device 228, e.g., such that there is just beginning to be some mutual repulsion.

In FIG. 3B, the distal end of tube 116 is closer to the coronary artery (CA), limited by the resulting increased repulsion, and has driven an anchor 26 into the annulus of the mitral valve (MV).

FIGS. 3C-E show subsequent similar steps in the process of driving in subsequent anchors 26, with device 228 being advanced through the coronary artery in concert with the movement of tube 116 around annulus 5, such that magnet 32 is positioned opposite each anchor as it is driven into the annulus. FIG. 3E shows the situation after all of anchors 26 anchored, and magnetic device 228 is ready to be withdrawn.

Reference is made to FIGS. 4A-C, which are schematic illustrations of a heart valve repair system 18, in accordance with some applications. System 18 comprises a vessel-protection device 250 that comprises a longitudinal member 252, and a magnet 254, disposed at a distal portion of the longitudinal member. Magnet 254 is oriented such that its magnetic axis ax2 is substantially orthogonal to longitudinal axis ax1 of longitudinal member 252. The distal portion of longitudinal member 252 is transluminally advanceable, along axis ax1, into the coronary artery. In some applications, the longitudinal member is one or more of a rod, wire, extension, tube, hypotube, strip, ribbon, shaft, etc.

System 18 is configured (e.g., primarily configured) to facilitate orienting anchor 26 and/or its anchor driver 114′ such that the anchor is driven into annulus 5 at a desirable angle of attack, e.g., with the anchor not pointing toward the coronary artery. However, for some applications, system 18 can be configured (e.g., modified) to also magnetically distance anchor 26 from the coronary artery, e.g., similarly to as described for systems 10, 11, and 12, mutatis mutandis.

A delivery tool 110′ is shown being used to deliver and anchor anchor 26. Delivery tool 110′ comprises an anchor driver 114′ that can comprise a flexible tube 116′ and a rod (e.g., rod 118, described hereinabove). The use of the suffix ′ in the reference numerals of delivery tool 110′ and its components is intended to indicate that they are similar, and serve similar functions, to delivery tool 110 and its components. Delivery tool 110′ can further comprise a catheter, such as catheter 112 (described hereinabove), via which driver 114′ can be advanced.

A distal portion of tube 116′ defines a tube axis ax3, along which anchor 26 is advanceable out of the tube. Delivery tool 110′ comprises a magnet 260 (i.e. magnet 260 is in addition to magnet 254), disposed at a distal portion of tube 116′. A magnetic axis ax4 of magnet 260 is substantially parallel with tube axis ax3—thus axes ax3 and ax4 are shown as coincident in FIGS. 4A-C.

FIG. 4A shows driver 114′ bringing anchor 26 toward annulus 5, and in doing so, bringing tube 116′ into proximity with device 250. As tube 116′ becomes closer to device 250 (and therefore the coronary artery), magnetic forces between magnets 254 and 260 (e.g. repulsion of the south pole (SP) of magnet 260 from the SP of magnet 254; attraction of the SP of magnet 260 toward the north pole (NP) of magnet 254; and/or attraction of the NP of magnet 260 toward the SP of magnet 254) biases (e.g., deflects) magnetic axis ax4 (and therefore tube axis ax3) toward being parallel with magnetic axis ax3. Because of the rotational orientation of device 250 within the coronary artery (which may be pre-planned), this biases the angle of attack of anchor 26 away from coronary artery (FIG. 4B). FIG. 4C shows driver 114′ contacting annulus 5 after magnetic axis ax4 has been further biased toward being parallel with magnetic axis ax3—and therefore further biasing the angle of attack away from the coronary artery. FIG. 4C also shows rod 118 driving anchor 26 out of tube 116′ and into annulus 5 at this angle of attack.

Tube 116′ can be configured to be sufficiently flexible to bend in response to the magnetic force between magnets 254 and 260, thereby facilitating the above-described biasing.

For some applications, at least one of magnets 254 and 260 is an electromagnet. For some such applications, the other of the magnets is a permanent magnet. For other such applications, both magnets are electromagnets.

Although only one magnet 254 is shown, for some applications device 250 comprises multiple magnets 254, e.g., distributed along the distal portion of longitudinal member 252. For example, such a device may be used to facilitate the anchoring of multiple anchors, e.g., as described with reference to FIGS. 2A-C, mutatis mutandis. For other applications, multiple anchors can be anchored by moving a single magnet 254 (e.g., by moving longitudinal member 252) along the coronary artery, e.g., as described with reference to FIGS. 3A-E, mutatis mutandis.

While the examples described hereinabove may relate to protecting the coronary artery using a vessel-protection device temporarily within the coronary artery to induce magnetic repulsion/deflection, the following examples relate to protecting the coronary artery using a vessel-protection device temporarily within the coronary to induce temporary mechanical repulsion/deflection of the coronary artery away from the site at which the anchor is to be anchored. However, it is to be noted that the scope of the present disclosure includes applications that combine one or more features of one or more of the vessel-protection devices described hereinabove (e.g., one or more magnetic-repulsion features) with one or more features of one or more of the vessel-protection devices described hereinbelow (e.g., one or more mechanical repulsion/deflection features), e.g., applications that utilize both temporary mechanical repulsion/deflection and magnetic repulsion.

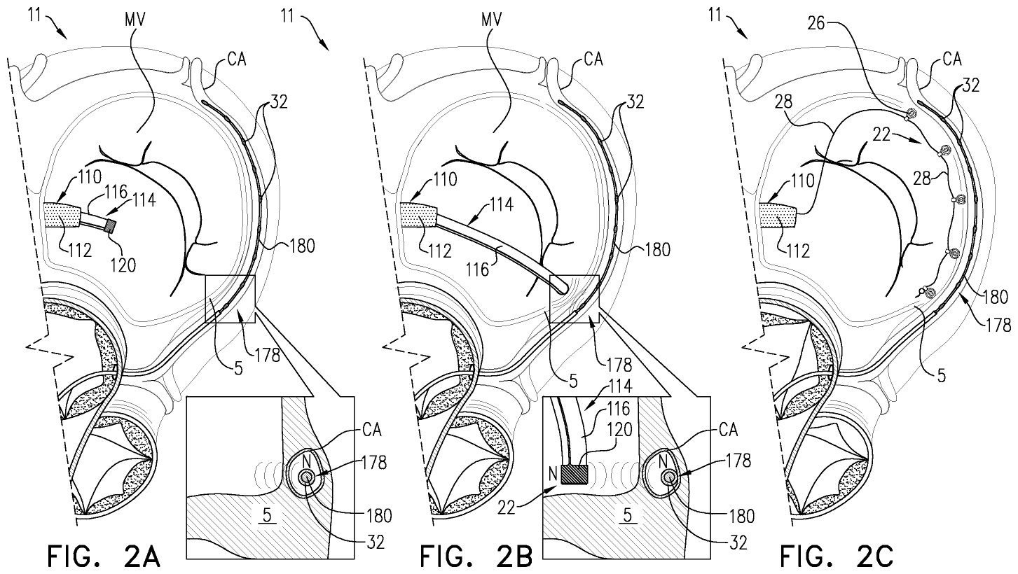

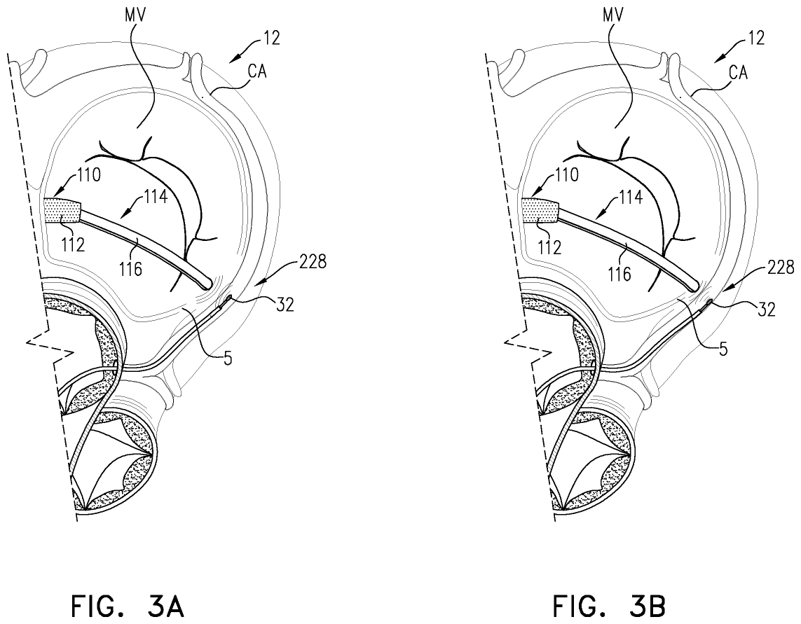

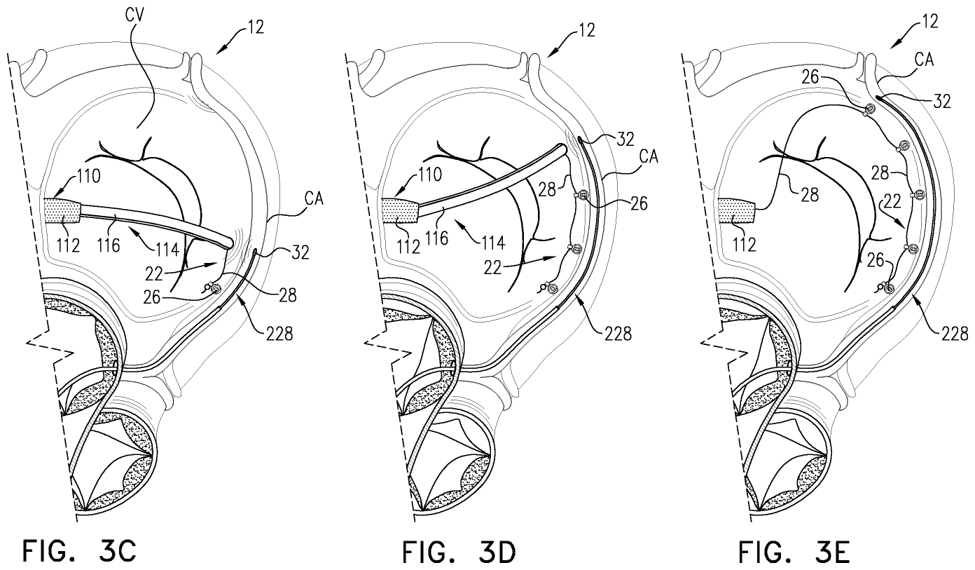

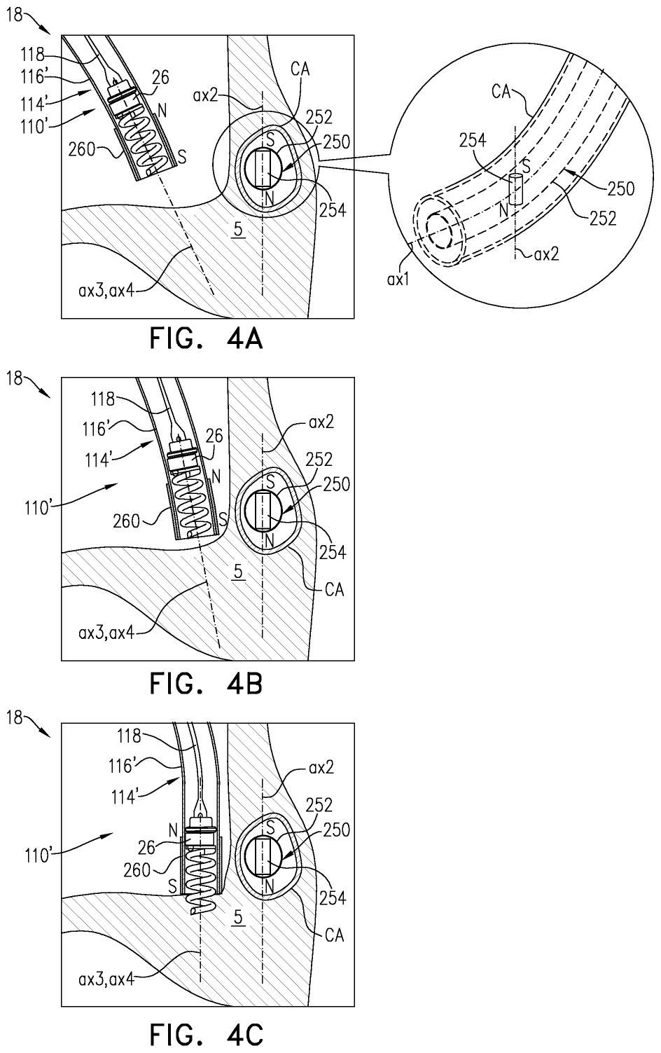

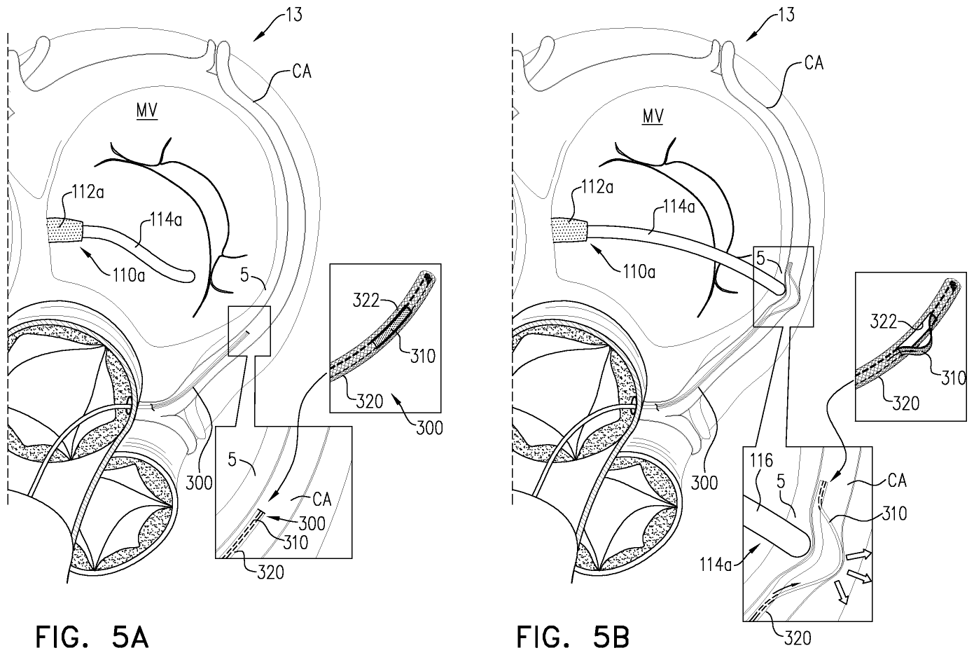

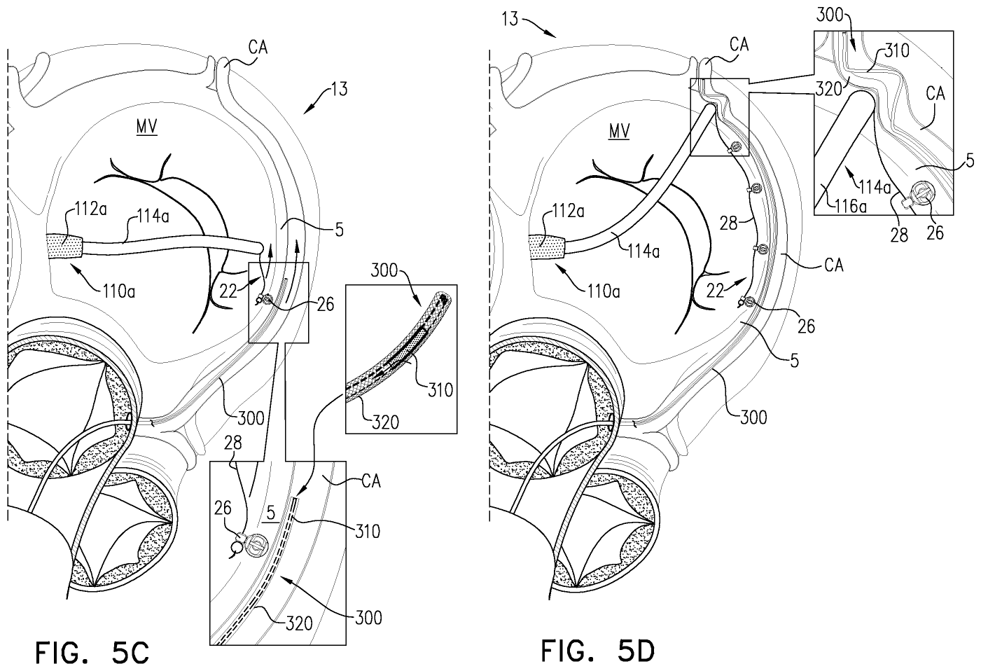

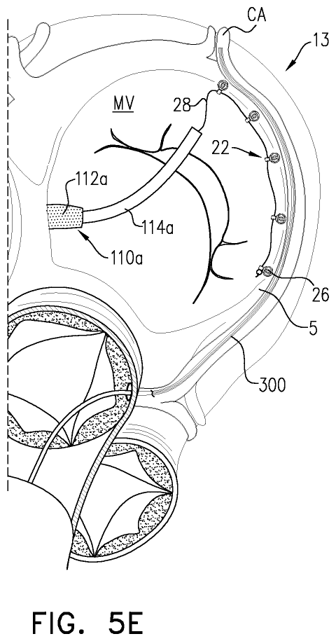

Reference is made to FIGS. 5A-E and 6A-C, which are schematic illustrations of heart valve repair systems 13 and 14, in accordance with some applications. System 13 comprises a vessel-protection device 300 that comprises a deflector 310, and system 14 comprises a vessel-protection device 400 that comprises a deflector 410. A delivery tool 110a is shown being used to deliver and anchor implant 22—including anchors 26 of the implant. Delivery tool 110a comprises a catheter 112a and an anchor driver 114a that can comprise a flexible tube 116a and a rod. The use of the suffix “a” in the reference numerals of delivery tool 110a and its components is intended to indicate that they are similar, and serve similar functions, to delivery tool 110 and its components.

Each of deflectors 310 and 410 has a distal portion that comprises an elastic or shape-memory material that biases the distal portion toward assuming a deflecting state in which the distal portion forms a bend or bulge. However, each of devices 300 and 400 also comprises a restraint (320 and 420, respectively) that constrains the distal portion in a non-deflecting state, e.g., for transluminal advancement into and within the coronary artery (CA). In the examples shown, restraint 320 of device 300 and restraint 420 of device 400 are tubes. However, it is to be noted that the scope of the disclosure includes other types of restraint. Elimination of the constraint, e.g., by exposing at least part of the distal portion from the restraint—allows the distal portion to move toward its deflecting state.

For some applications, in the deflecting state of the deflector, the deflector deflects the coronary artery away from the annulus, in particular away from the site at which an anchor 26 is to be anchored. For some applications, a deflection of at least 3 mm and/or no more than 10 mm of the coronary artery may provide a sufficient spacing between the coronary artery and the anchor being anchored. For example, deflectors 310 and 410 may deflect the coronary artery by at least 3 mm (e.g. by at least 5 mm) and/or by no more than 10 mm (e.g. by no more than 8 mm, such as by no more than 7 mm), such as by between 5 mm and 8 mm, such as by approximately 6 mm. For some applications, deflector 310 and/or 410 may provide an even greater magnitude of deflection.

For some applications, vessel-protection device(s) 300 and/or 400 (e.g. deflector(s) 310 and/or 410) are radiopaque and/or echogenic, so that intraprocedural imaging can allow an operator to determine whether suitable deflection of the coronary artery has been achieved (e.g. prior to anchoring anchors 26).

Once implantation of anchors 26 is complete, the deflector is typically returned to its non-deflecting state, allowing for the coronary artery to return toward its original position and/or form.

For some applications, and as shown, deflector 310 (or at least the distal portion thereof) comprises a wire or ribbon, e.g., having a consistent shape along its length.

FIG. 5A illustrates device 300 being advanced in the coronary artery. FIG. 5B shows the distal portion of deflector 310 bulging out of a lateral window 322 of restraint 320, and away from mitral valve annulus 5, thereby deflecting the coronary artery away from the annulus, in particular away from the site at which an anchor 26 is to be anchored. This may be achieved by deflector 310 being advanced (e.g., pushed distally) relative to restraint 320, thereby feeding the deflector out of window 322. In some applications, this may be achieved by releasing a proximal portion of the deflector to allow the distal portion of the deflector to elastically move toward its deflecting state, pulling the proximal portion of the deflector distally. Distal from window 322 (e.g., at the distal end of restraint 320 and/or at the distal end of deflector 310), the deflector can be fixed to restraint 320.

FIG. 5C shows deflector 310 having been returned to its delivery state (e.g., by pulling on the deflector relative to restraint 320), and tool 110a and device 300 being moved onward for anchoring of another anchor 26. FIG. 5D shows anchoring of a final anchor 26, and FIG. 5E illustrates the situation prior to tensioning of tether 28 and withdrawal of vessel-protection device 300.

Device 400 can be similar to device 300, except as noted. For example, deflector 410 increases its span laterally (e.g. to contact opposing sides of the coronary artery) to stabilize it within the coronary artery, e.g., reducing a likelihood that it will flip. This lateral spanning may be achieved by deflector 410 defining a forked end, e.g., as shown. A further difference is that deflector 410 is typically exposable from a distal end of restraint 420, e.g., by advancing the deflector and/or retracting the restraint.

As seen in the upper enlargement of FIG. 6B, the distal portion of deflector 410 can have a spider-like form or can resemble a four-legged animal with the back 412 of the animal deflected away from the site at which the anchor 26 is to be anchored.

FIG. 6A shows device 300 being advanced to a first location while constrained in its delivery (e.g., flattened and/or compressed) state, and FIG. 6B shows a first anchor 26 being anchored at that first location while deflector 410 is in its deflected state. FIG. 6C shows deflector 410 having been returned to its delivery state (e.g., by retracting the deflector into restraint 420), and tool 110a and device 400 being moved onward for anchoring of another anchor 26.

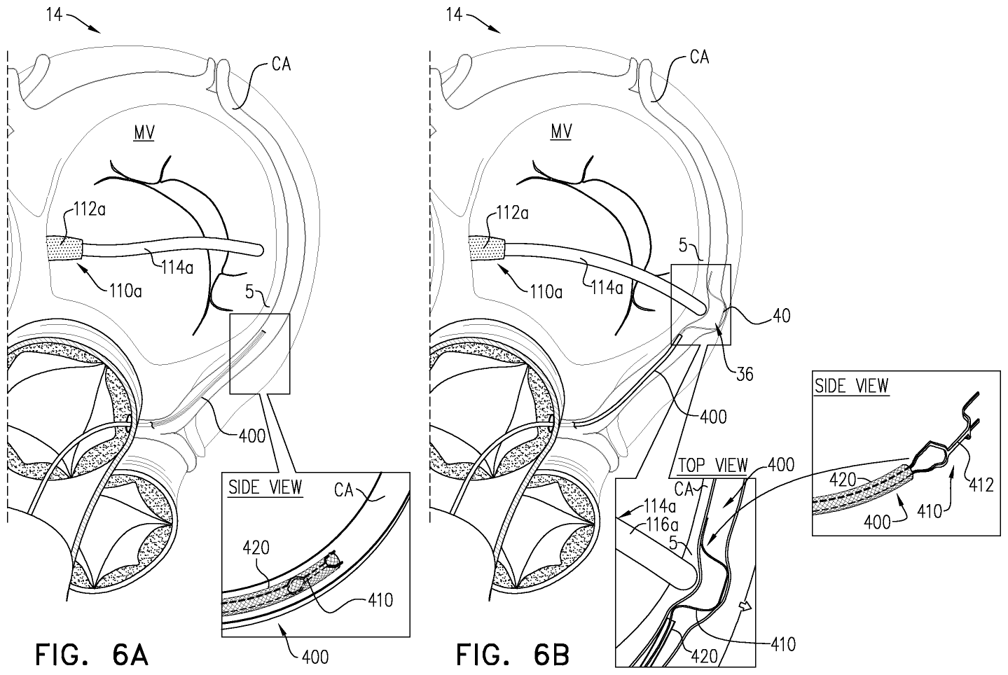

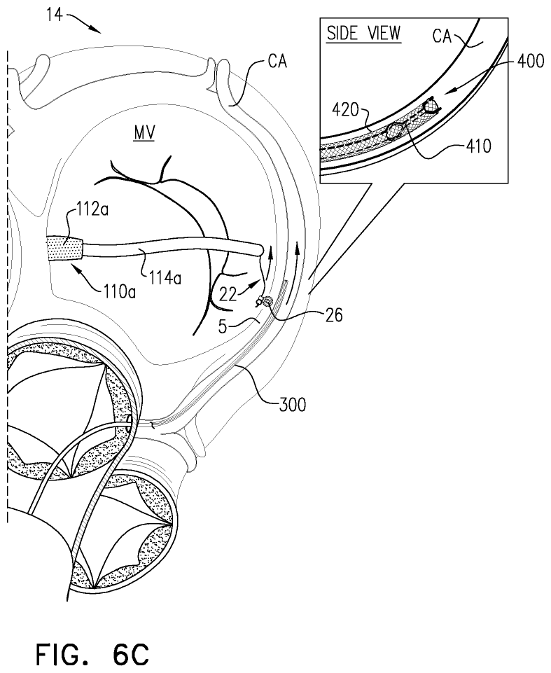

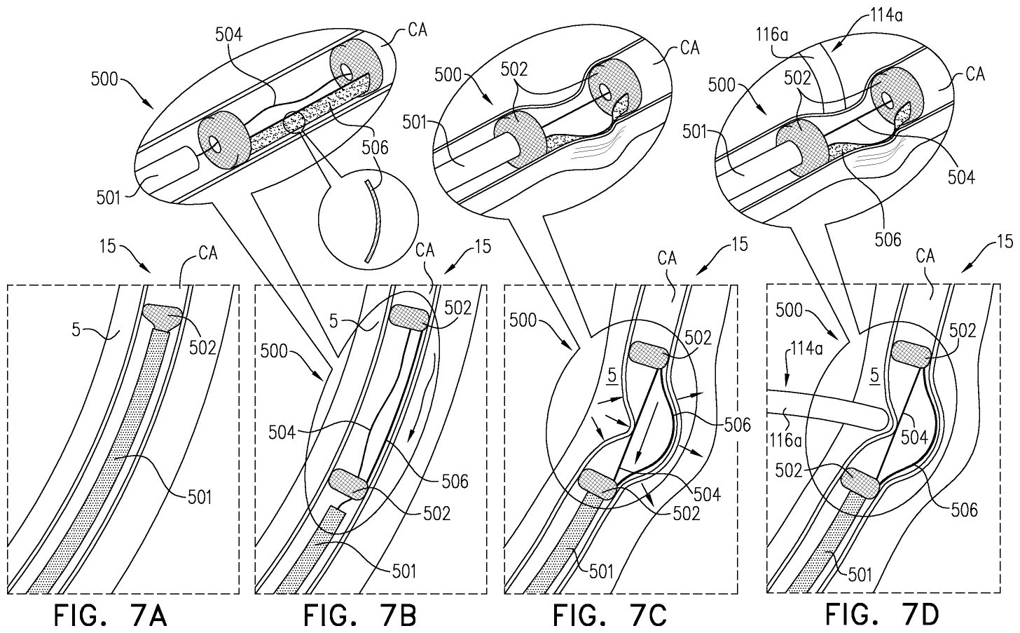

Reference is made to FIGS. 7A-D, which are schematic illustrations of another heart valve repair system 15, which comprises a vessel-protection device 500, in accordance with some applications. Device 500 is configured to produce a bend or curve in a coronary artery (CA) in order to move part of the coronary artery away from the site at which an anchor 26 is to be anchored. Device 500 includes a pair of grips 502, each optionally having a hollow inner portion (e.g., the grips being annular or tubular) in order to allow bloodflow therethrough. Device 500 can further comprise a tether 504 connected to at least one of grips 502, such as to a distal one of the grips.

Grips 502 are configured to be secured within the coronary artery (CA), such as by expansion of the grips against the inner wall of the coronary artery. For example, each grip 502 can comprise a stent-like structure, a coiled structure (e.g., a torsion spring or a helical spring), or an inflatable element.

Grips 502 are temporarily secured within the coronary artery (CA) distally and proximally from the location adjacent to where anchor 26 is to be anchored (FIGS. 7A-B). As illustrated in FIG. 7C, tether 504 is then tensioned to draw grips 502 toward each other (e.g. to draw the distal grip towards the proximal grip, thereby compressing the portion of the coronary artery therebetween) in order to form slack in the portion of the coronary artery between grips 502. This may be facilitated by providing a reference force (e.g., a pushing force) on the proximal one of grips 502, e.g., using a tube 501 via which the grips were initially introduced.

Tensioning tether 504 such that grips 502 move towards each other may cause a deflector (e.g., a band) 506, disposed between grips 502, bulges laterally, to push the slackened portion of the coronary artery laterally, away from the site at which the anchor 26 is to be anchored. For some applications, deflector 506 is itself biased to bulge in this manner, e.g., due to elasticity or shape memory. For some applications, force applied to deflector 506 causes it to bulge. For example, and as shown, deflector 506 can be attached at each of its ends to a respective one of grips 502, such that the drawing of the grips together causes the deflector to bulge. For some applications, and as shown, deflector 506 is an integral component of device 500, e.g., with the device provided with the deflector pre-attached to grips 502.

For some applications, deflector 506 is introduced separately from grips 502, e.g., after the slack on the artery has been formed. For some such applications, deflector 506 can be similar to other deflectors described herein, and/or grips 502 and tether 504 can be used to augment the functionality of other deflector-based vessel-protection devices described herein, mutatis mutandis.

FIG. 7D shows driver 114a anchoring an anchor 26 to the annulus while the coronary artery (CA) remains deflected away from the annulus by device 500.

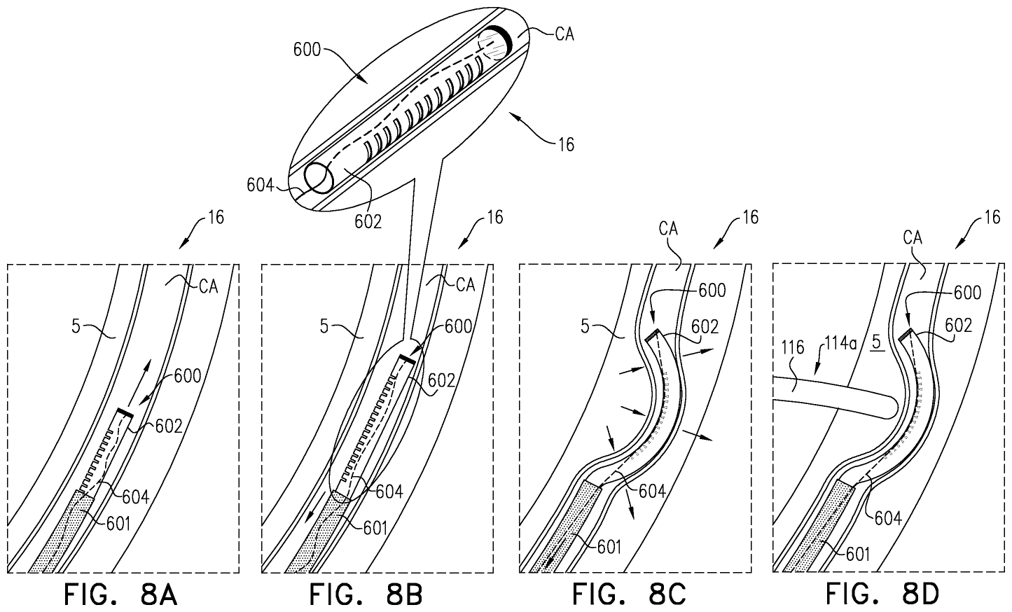

Reference is now made to FIGS. 8A-D and 9A-D, which are schematic illustrations of heart valve repair systems 16 and 17, in accordance with some applications. Each of systems 16 and 17 comprises a vessel-protection device that comprises a tether and a deflector that can be bent via tensioning of the tether, so that the deflector bulges away from the site at which an anchor 26 is to be anchored.

System 16 comprises a vessel-protection device 600 that comprises a tether 604 and a deflector 602 that comprises (e.g., is) a flexible tube. Tether 604 is fixed to a distal part of the tube, and extends proximally along (e.g., through) the tube. Tensioning of tether 604 (e.g., while applying a reference force to a proximal part of deflector 602) causes the tube to bend (e.g., to curve), thereby bulging away from the site at which an anchor 26 is to be anchored (FIG. 8C). Deflector 602 can have a series of lateral slots therein, to facilitate unidirectional bending.

Device 600 can be advanced into the coronary artery (CA) via a tube 601 (FIG. 8A) that is then withdrawn to expose deflector 602 (FIG. 8B). FIG. 8C shows tensioning of tether 604 to cause deflector 602 to bend, thereby bulging away from the site at which an anchor 26 is to be anchored. FIG. 8D shows driver 114a anchoring an anchor 26 to the annulus while the coronary artery (CA) remains deflected away from the annulus by device 600.

System 17 comprises a vessel-protection device 700 that comprises a tether 704 and a deflector 702 that comprises multiple (e.g., two or three) articulated sections 706 in series. Tether 704 is fixed to a distal part of the deflector, and extends proximally along (e.g., alongside) the deflector. Tensioning of tether 704 (e.g., while applying a reference force to a proximal part of deflector 702) causes the deflector to bend (e.g., causes sections 706 to articulate), thereby bulging away from the site at which an anchor 26 is to be anchored (FIG. 9C). Deflector 702 can have a hinge 708, a flexure joint, or another type of joint between adjacent sections 706, to facilitate articulation.

Device 700 can be advanced into the coronary artery (CA) via a tube 701 (FIG. 9A) that is then withdrawn to expose deflector 702 (FIG. 9B). FIG. 9C shows tensioning of tether 704 to cause deflector 702 to bend, thereby bulging away from the site at which an anchor 26 is to be anchored. FIG. 9D shows driver 114a anchoring an anchor 26 to the annulus while the coronary artery (CA) remains deflected away from the annulus by device 700.

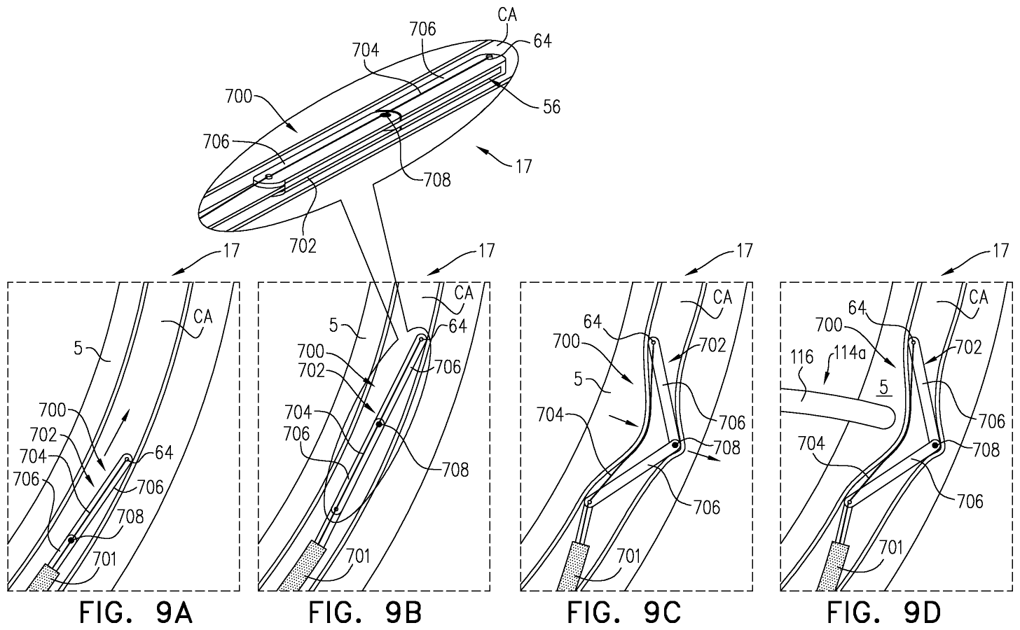

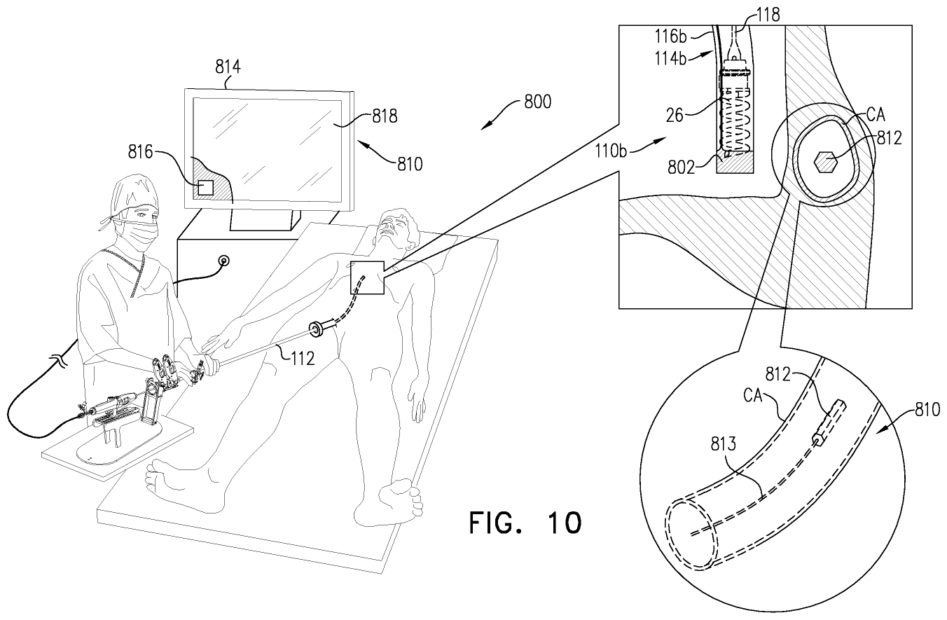

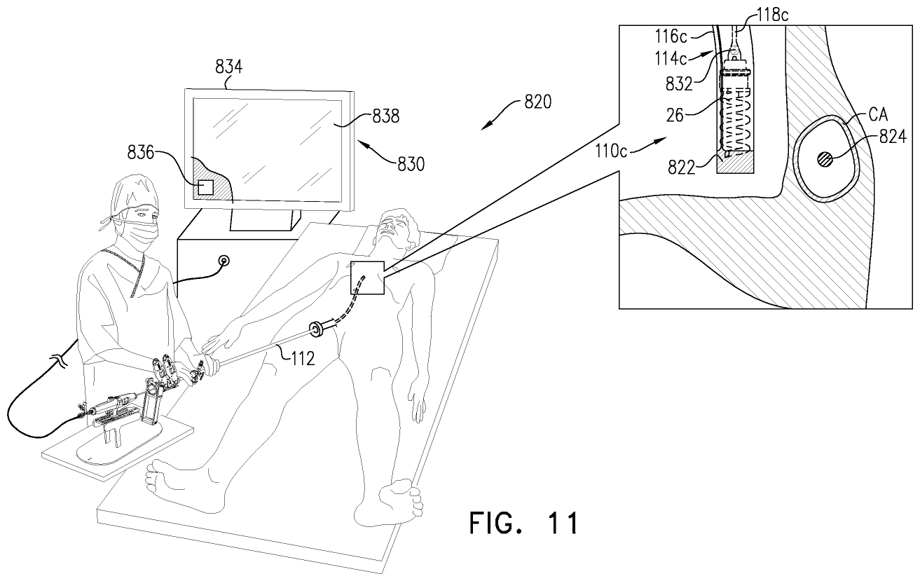

Reference is now made to FIGS. 10-11, which are schematic illustrations of heart valve repair systems 800 and 820, respectively, in accordance with some applications. Whereas some of the systems described hereinabove utilize magnetism to repel and/or deflect an anchor and/or its anchor driver away from the coronary artery, each of systems 800 and 820 is configured to utilize magnetism to indicate to the operator a position of the anchor and/or its anchor driver within the heart, e.g., a proximity of the anchor and/or anchor driver to the coronary artery.

System 800 comprises a delivery tool 110b, which is used to deliver and anchor one or more anchors 26. Tool 110b comprises an anchor driver 114b that comprises a flexible tube 116b, transluminally advanceable into the atrium, and a rod 118 (e.g., rod 118, described hereinabove), configured to drive an anchor 26 out of the distal portion of the tube and into annulus 5. The use of the suffix “b” in the reference numerals of delivery tool 110b and its components is intended to indicate that they are similar, and serve similar functions, to delivery tool 110 and its components. Delivery tool 110b can further comprise a catheter, such as catheter 112 (described hereinabove), via which driver 114b can be advanced.

Anchor driver 114b can comprise a magnet 802, disposed at a distal portion of tube 116b.

System 800 further comprises a position-detecting assembly 810, comprising a microelectromechanical system magnetic field sensor (MEMS-MFS) 812, and an extracorporeal interface 814. MEMS-MFS 812 is transluminally advanceable into the coronary artery (e.g., mounted on a longitudinal member 813) and is configured to sense a magnetic field of magnet 802.

Assembly 810 is configured to derive, from the sensed magnetic field, information regarding a position (e.g. a relative position) of the distal portion of tube 116b within the heart. More specifically, assembly 810 can be configured to derive, from the sensed magnetic field, information regarding a proximity of magnet 802 (and thereby of the distal portion of tube 116b) to MEMS-MFS 812, and thereby to the coronary artery within which the MEMS-MFS is disposed. Assembly 810 can comprise circuitry 816 configured to facilitate or perform (e.g., to process) this derivation. Circuitry 816 can be in electronic communication with (e.g., can be electronically connected to) MEMS-MFS 812 via longitudinal member 813.

Assembly 810 can provide, e.g., to the operator, the derived information, e.g., visually via a display 818, and/or audibly. The provided information can include and/or be combined with other navigation information, e.g., by, on an image of the heart, representing the position of tube 116b (e.g., relative to the coronary artery), based on the derived information regarding the proximity of magnet 802 to MEMS-MFS 812.

System 820 comprises a delivery tool 110c, which is used to deliver and anchor one or more anchors 26. Tool 110c comprises an anchor driver 114c that comprises a flexible tube 116c, transluminally advanceable into the atrium, and a rod 118c, (e.g., similar to rod 118, except where noted), configured to drive an anchor 26 out of the distal portion of the tube and into annulus 5. The use of the suffix “c” in the reference numerals of delivery tool 110c and its components is intended to indicate that they are similar, and serve similar functions, to delivery tool 110 and its components. Delivery tool 110c can further comprise a catheter, such as catheter 112 (described hereinabove), via which driver 114c can be advanced.

Anchor driver 114c comprises a magnet 822, that can be disposed at a distal portion of tube 116b.

System 820 further comprises a position-detecting assembly 830, comprising a microelectromechanical system magnetic field sensor (MEMS-MFS) 832, and an extracorporeal interface 834. MEMS-MFS 832 is mounted on delivery tool 110c, such as on rod 118c (e.g., on a distal portion of the rod), and is transluminally advanceable into the atrium, from where it is configured to sense a magnetic field of magnet 822.

System 820 also comprises a longitudinal member 824, which is advanceable into the coronary artery. Longitudinal member 824 can be metallic, and can simply be a wire, such as a guidewire. In some applications, the longitudinal member is one or more of a rod, wire, extension, tube, hypotube, strip, ribbon, shaft, etc. Although FIG. 11 shows only a cross-section of longitudinal member 824, it is to be understood that the longitudinal member defines such a cross-section along at least part of its length. For example, longitudinal member 824 may resemble longitudinal member 813.

Assembly 830 is configured to derive, from the sensed magnetic field, information regarding a position of the distal portion of tube 116c within the heart. More specifically, assembly 830 can be configured to derive, from the sensed magnetic field, information regarding a proximity of a distal portion of tube 116c to longitudinal member 824, and thereby to the coronary artery within which the longitudinal member is disposed. This can be accomplished by MEMS-MFS 832 detecting, in the magnetic field of magnet 822, distortions caused by longitudinal member 824 (e.g., passively, merely by the presence of the longitudinal member). Assembly 830 can comprise circuitry 836 configured to facilitate or perform (e.g., to process) this derivation. Circuitry 836 can be in electronic communication with (e.g., can be electronically connected to) MEMS-MFS 832 via rod 118c.

Assembly 830 can provide, e.g., to the operator, the derived information, e.g., visually via a display 838, and/or audibly. The provided information can include and/or be combined with other navigation information, e.g., by, on an image of the heart, representing the position of tube 116c (e.g., relative to the coronary artery), based on the derived information regarding the proximity to longitudinal member 824.

It will be understood that the above-described derivation can be implemented by computer program instructions. These computer program instructions can be provided to and/or performed by circuitry 816 and/or 836.

Circuitry 816 and/or 836 can comprise a processor such as a general-purpose processor (e.g., of a general-purpose computer), a special-purpose processor (e.g., of a special-purpose computer), an application-specific integrated circuit (ASIC), or other programmable data processing apparatus. The instructions that execute via the circuitry create means for implementing the above-described derivations.

The assembly 810, 830 may include a computer-readable medium. In some implementations, the circuitry 816, 836 may include the computer-readable medium or may otherwise be directed by the computer-readable medium to perform the computer program instructions. The computer program instructions can be stored in a computer-readable medium (e.g., a non-transitory computer-readable medium) and can direct circuitry 816 and/or 836 to function in a particular manner, such that the instructions stored in the computer-readable medium produce an article of manufacture including instruction means which implement the function(s)/act(s) described in the present disclosure. The computer program instructions can also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the function(s)/act(s) described in the present disclosure.

For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. The computer-usable or computer readable medium can be a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

Network adapters can be coupled to circuitry 816 and/or 836 to enable the circuitry to become coupled to other circuitry, processors, or storage devices through intervening private or public networks.

Computer program code for carrying out operations of the present disclosure may be written in any combination of one or more programming languages, including an object-oriented programming language such as Java, Smalltalk, C++ or the like and conventional procedural programming languages, such as the C programming language or similar programming languages.

Although the disclosure hereinabove shows the left coronary artery as being the coronary blood vessel into which vessel-protection device are temporarily placed, it is to be noted that the scope of the invention includes temporary placement of the various devices, mutatis mutandis, into another coronary artery (e.g., the right coronary artery) or into a coronary vein (e.g., the coronary sinus).

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.). Furthermore, the scope of the present disclosure includes, for some applications, sterilizing any of the various systems, devices, apparatuses, etc. in this disclosure.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, anthropomorphic ghost, simulator (e.g., with the body parts, tissue, etc. being simulated), etc.

1. A method, comprising:
transluminally advancing a vessel-protection device into a blood vessel; and
using a delivery tool, driving an anchor into tissue adjacent the blood vessel while temporarily distancing the blood vessel from the anchor using the vessel-protection device disposed within the blood vessel.

2. The method according to claim 1, further comprising, subsequently to driving the anchor into the tissue, removing the vessel-protection device from the blood vessel.

3. The method according to claim 1, wherein the tissue is tissue of a atrium of a heart, and wherein driving the anchor into the tissue comprises using a delivery tool disposed within the atrium to drive the anchor into the tissue.

4. The method according to claim 1, wherein the tissue is tissue of a ventricle of a heart, and wherein driving the anchor into the tissue comprises using a delivery tool disposed within the ventricle to drive the anchor into the tissue.

5. The method according to claim 1, wherein:
the anchor is a first anchor of an implant, and
driving the anchor into the tissue comprises driving the first anchor of the implant into the tissue at a first site while distancing the blood vessel from the first anchor using the vessel-protection device disposed within the blood vessel in proximity to the first site, and
the method further comprises:
subsequently to driving the first anchor into the tissue at the first site, moving the vessel-protection device within the blood vessel from being in proximity to the first site to being in proximity to a second site, and
driving a second anchor of the implant into the tissue at the second site while distancing the blood vessel from the second anchor using the vessel-protection device disposed within the blood vessel in proximity to the second site.

6. The method according to claim 5, wherein the implant is an adjustable implant including a tether coupled to the anchor, and wherein the method further comprises adjusting the implant by tensioning the tether subsequently to driving the anchor into the tissue.

7. The method according to claim 1, wherein the tissue is tissue of a annulus of a heart valve, and the blood vessel is a coronary blood vessel in proximity to the annulus, and wherein driving the anchor into the tissue comprises driving the anchor into the annulus.

8. The method according to claim 7, wherein the anchor is an anchor of an annuloplasty implant, and wherein driving the anchor into the annulus comprises driving the anchor of the annuloplasty implant into the annulus.

9. The method according to claim 1, wherein driving the anchor into the tissue comprises driving the anchor into the tissue using a delivery tool while distancing the blood vessel from the anchor by applying a mechanical force using the vessel-protection device disposed within the blood vessel.

10. The method according to claim 9, wherein the vessel-protection device includes a deflector, and wherein applying the mechanical force using the vessel-protection device comprises temporarily pushing a part of the blood vessel away from the tissue using the deflector.

11. The method according to claim 10, wherein pushing the part of the blood vessel away from the tissue using the deflector comprises pushing the part of the blood vessel away from the tissue by temporarily transitioning the deflector into a deflecting state in which the deflector bulges away from the tissue.

12. The method according to claim 11, wherein:
the deflector is biased toward assuming the deflecting state,
transluminally advancing the vessel-protection device into the blood vessel comprises transluminally advancing the deflector into the blood vessel while the deflector is constrained in a non-deflecting state, and
transitioning the deflector into the deflecting state comprises eliminating the constraint such that the deflector automatically responsively transitions toward the deflecting state.

13. The method according to claim 11, wherein:
the vessel-protection device includes a tether that extends proximally from the deflector, and
transitioning the deflector into the deflecting state comprises transitioning the deflector into the deflecting state by tensioning the tether.

14. The method according to claim 13, wherein transitioning the deflector into the deflecting state comprises transitioning the deflector into the deflecting state by tensioning the tether such that the deflector curves.

15. The method according to claim 13, wherein transitioning the deflector into the deflecting state comprises transitioning the deflector into the deflecting state by tensioning the tether such that the deflector articulates.

16. The method according to claim 1, wherein:
the blood vessel is of a heart,
the heart has a valve adjacent a chamber, the valve having a annulus, the tissue being tissue of a annulus of the valve, the blood vessel being in proximity to the annulus, and
driving the anchor into the tissue comprises driving the anchor into the tissue using a delivery tool while distancing the blood vessel via magnetic repulsion using the vessel-protection device disposed within the blood vessel.

17. The method according to claim 16, wherein distancing the blood vessel via magnetic repulsion comprises distancing the blood vessel via magnetic repulsion between the vessel-protection device disposed within the blood vessel and the anchor disposed within the chamber.

18. The method according to claim 16, wherein distancing the blood vessel via magnetic repulsion comprises distancing the blood vessel via magnetic repulsion between the vessel-protection device disposed within the blood vessel and the delivery tool disposed within the chamber.

19. The method according to claim 1, further comprising sterilizing the vessel-protection device, the anchor or the delivery tool.

20. A method, comprising:
transluminally advancing a vessel-protection device into a simulated blood vessel; and
using a delivery tool, driving an anchor into simulated tissue adjacent the simulated blood vessel while temporarily distancing the simulated blood vessel from the anchor using the vessel-protection device disposed within the simulated blood vessel.

21. A method, for treating a heart of a subject, the heart having a chamber and a coronary blood vessel in adjacent the chamber, the method comprising:
transluminally advancing an anchor driver into the chamber, the anchor driver including:
a flexible tube, having a distal portion that:
has a tube axis,
is transluminally advanceable into the chamber, and
has a distal opening;
a first magnet, disposed at the distal portion of the tube, and having a first magnetic axis that is substantially parallel with the tube axis; and
a rod, disposed within the tube;
transluminally advancing a vessel-protection device into the coronary blood vessel, the vessel-protection device including:
a longitudinal member, having a distal portion that has a longitudinal axis and that is transluminally advanceable into the coronary blood vessel; and
a second magnet, disposed at the distal portion of the longitudinal member, and having a second magnetic axis that is substantially orthogonal to the longitudinal axis;
bringing the distal portion of the tube, within the chamber, into proximity with the vessel-protection device within the coronary blood vessel, such that a magnetic force between the first magnet and the second magnet biases the tube axis toward being parallel with the second magnetic axis; and
while the magnetic force continues to bias the tube axis toward being parallel with the second magnetic axis, driving an anchor out of the distal opening and into tissue of the heart.
